(19) United States
(12) Patent Application Publication
(10) Pub. No.: US 2025/0240178 A1
(43) Pub. Date: Jul. 24, 2025

(54) EDIBLE UNCLONABLE FUNCTIONS

(71) Applicant: Purdue Research Foundation, West Lafayette, IN (US)

(72) Inventors: Young L Kim, West Lafayette, IN (US); Jung Woo Leem, West Lafayette, IN (US)

(73) Assignee: Purdue Research Foundation, West Lafayette, IN (US)

(21) Appl. No.: 18/986,156

(22) Filed: Dec. 18, 2024

Related U.S. Application Data
(62) Division of application No. 17/768,289, filed on Apr. 12, 2022, now Pat. No. 12,184,796.
(60) Provisional application No. 62/915,666, filed on Oct. 16, 2019.
(60) Provisional application No. 62/915,667, filed on Oct. 16, 2019.

(51) Int. Cl. H04L 9/32; A61K 47/42; G06Q 30/018

(52) U.S. Cl. CPC H04L 9/3278; A61K 47/42; G06Q 30/018

(57) ABSTRACT

A method of fabricating physically unclonable function includes constructing plasmid vector DNA for silkworm transgenesis to thereby generate one or more fluorescent silk proteins, removing sericin from the one or more fluorescent silk proteins, cutting the sericin-removed product into a plurality of pieces, dissolving the cut pieces with a solution, filtering the dissolved solution, dialyzing the filtered solution, centrifuging the dialyzed solution to thereby provide a regenerated silk fibroin solution, freeze-drying the regenerated silk fibroin solution, mechanically grinding the freeze-dried silk fibroin into granular silk microparticles, sieving the mechanically ground silk fibroin to generate fluorescent silk microparticles, generating an admixture of the fluorescent silk microparticles, broadcasting the admixture of the fluorescent silk microparticles to the surface of a polystyrene Petri dish thereby generating a silk film that are adapted to be attached to a pharmaceutical, wherein the silk film includes random distribution of the one or more fluorescent silk proteins.

### CROSS-REFERENCE TO RELATED APPLICATIONS

The present patent application is a Divisional Application of a 35 U.S.C. § 371 Nationalization Application S/N 17/768,289 which claims the priority benefit of the International Patent Application Serial No. PCT/US20/46580 filed Aug. 15, 2020, which is related to and claims the priority benefit of U.S. Provisional Patent Application Ser. No. 62/915,667 filed 16-OCT-2019 entitled “EDIBLE UNCLONABLE FUNCTIONS”, and U.S. Provisional Patent Application Ser. No. 62/915,666 filed 16 Oct. 2019 entitled “IMAGE PROCESSING AND AUTHENTICATION OF EDIBLE UNCLONABLE FUNCTIONS”, the contents of each of which are hereby incorporated by reference in its entirety into the present disclosure.

### STATEMENT REGARDING GOVERNMENT FUNDING

This invention was made with government support under FA2386-17-1-4072 awarded by US Air Force Office of Scientific Research. The government has certain rights in the invention.

### TECHNICAL FIELD

The present disclosure generally relates to counterfeit measures, and in particular, to an arrangement concerning an edible unclonable function counterfeit measure.

### BACKGROUND

This section introduces aspects that may help facilitate a better understanding of the disclosure. Accordingly, these statements are to be read in this light and are not to be understood as admissions about what is or is not prior art.

Counterfeit medicines have become ubiquitous, presenting myriad problems. This problem of counterfeit medicines is not a new one, but is becoming a tremendous burden to society in all countries. While ‘fake’ pharmaceutical products can be explicitly categorized into a plurality of categories including substandard, falsified, counterfeit, and diverted ones, they are all often referred to, as a single group, counterfeit medicines. They pose a significant threat to patient safety and public health as well as cause heavy economic losses in developed and less developed countries. As a devastating example, counterfeit drugs for malaria and pneumonia treatments cause estimated 250,000 child deaths each year. Counterfeit medicines of both lifestyle drugs (e.g. treatments for erectile dysfunction) and lifesaving drugs (e.g. treatments for cancer, malaria, diabetes, etc.) are increasingly being produced in developed and developing countries, in part due to the increased public use of online pharmacies. In addition, as an infringement of intellectual properties, scientific innovations and financial rewards in pharmaceutical companies are undermined by the widespread counterfeiting of medicine. The health and economic consequences of counterfeit medicines are far more serious in low- and middle-income countries. It is estimated that counterfeit medicines account for 10% of the global pharmaceutical trade and more than 20-30% of all medicines in Africa, Asia, and the Middle East.

There are a variety of approaches for detecting counterfeit medicines and for offering possible solutions for reducing the threat. Traditionally, analytical chemistry and spectroscopy technologies have been used to identify counterfeit medicines by detecting chemical signatures of major ingredients. However these techniques require sophisticated and expensive machines and have limited accuracy based on recognizing of such main ingredients. Other techniques include marking and printing on the medicine surface at various levels of resolution using lasers and other proprietary technologies, which modify the outer surface or coating of tablets or capsules, however, this technique is prone to duplication by counterfeiters. Recently, digital anti-counterfeit technologies have played a more significant role in authentication and supply chain. Package-level barcodes and radio frequency identification (RFID) are commonly used for instantaneous remote authentication. Several mobile technologies have been introduced for authentication services, track and trace solutions, and medicine recognitions. Detrimentally, such authentication and security techniques are symmetric; that is, if illegitimate manufacturers or sellers have access to the same techniques, it would be possible for them to create clones. An ideal authentication technology should be asymmetric with a form of on-dose authentication which can be directly swallowed and digestible. Specifically, on-dose (or in-dose) authentication means that every individual pill or dose is verified as genuine in the absence of packaging. Even if the original packaging is not retained by pharmacists or patients, the possibility of ingestion of counterfeit medicines is substantially eliminated. Indeed, the packaging information is often unavailable; pills are sold in small quantities and individual strips dispensed by pharmacists. On-dose authentication maximally reduces the opportunity for illegitimate sellers to use expired, counterfeit, or substandard drugs.

In this respect, a few promising technologies have recently been introduced with the potential of digital authentication, including digitally encoded polymers, QR-coded microtaggants and advanced wrinkle-based tags, QR code printing of active pharmaceutical ingredients, encoded-multifunctional hydrogel microparticles, large-scale microparticle arrays, encoded metal nanomaterials, and silica microtags. However, such materials are often not ideal from an oral intake safety perspective. These approaches rely on biocompatible and biodegradable yet exogenous materials, such as polystyrene, cellulose-acetate-phthalate (CAP), poly(lactic-co-glycolic acid) (PLGA), poly(ethylene glycol) (PEG), poly(ethylene glycol) diacrylate (PEGDA), silver, gold, and silica. It should be noted that foreign and nano-sized food additives could potentially have hazardous and adverse (e.g. carcinogenic and cytotoxic) effects, which currently have resulted in limited utilizations. In addition, such simple tagging technologies could be vulnerable to attackers, due to the limited security level.

Therefore, there is an unmet need for a novel approach to provide unclonable security for pharmaceuticals to combat the widespread availability of counterfeits.

### SUMMARY

A method of generating a physically unclonable function for pharmaceutical authentication is disclosed. The method includes generating an edible physically unclonable function (PUF), affixing the edible PUF onto a pharmaceutical in a random distribution, and generating a cryptographic key based on the randomly distributed and affixed edible PUF.

A method of fabricating and applying a physically unclonable function to a pharmaceutical is also disclosed. The method includes constructing plasmid vector DNA for silkworm transgenesis to thereby generate one or more fluorescent silk proteins. The method also includes removing sericin from the one or more fluorescent silk proteins. Furthermore, the method includes cutting the sericin-removed one or more fluorescent silk proteins into a plurality of pieces. Additionally, the method includes dissolving the cut pieces with a solution, filtering the dissolved solution, dialyzing the filtered dissolved solution, and centrifuging the dialyzed solution to thereby provide a regenerated silk fibroin solution. In addition, the method includes freeze-drying the regenerated silk fibroin solution, mechanically grinding the freeze-dried silk fibroin into granular silk microparticles, sieving the mechanically ground silk fibroin to generate fluorescent silk microparticles, and generating an admixture of the fluorescent silk microparticles. Furthermore, the method includes broadcasting the admixture of the fluorescent silk microparticles to the surface of a polystyrene Petri dish thereby generating a silk film that are adapted to be attached to a pharmaceutical. The silk film includes random distribution of the one or more fluorescent silk proteins.

### BRIEF DESCRIPTION OF DRAWINGS

FIG. 1a is a schematic of application of an edible physically unclonable functions (PUFs) for use with pharmaceuticals.

FIG. 1b shows schematics of photoluminescent properties of fluorescent silk proteins that are used to realize multiple challenge-response pairs in an edible PUF platform for heightened security according to the present disclosure.

FIG. 1c is a scanning electron microscope (SEM) output of an edible PUF device in which fluorescent silk microparticles are embedded in a thin silk film.

FIG. 1d is an emission spectra of particulate eCFP, eGFP, eYFP and mKate2 silk which cover a relatively broad wavelength range in the visible light, while the emission peak positions are not overlapped among others.

FIG. 1e provides confocal fluorescence microscopy images of the corresponding fluorescent silk microparticles under excitation of 405, 458, 514, or 561 nm for eCFP, eGFP, eYFP and mKate2 silk, respectively.

FIG. 2 is a transformation vector of p3xP3-DsRed2-FibH-(eCFP, eGFP, eYFP, or mKate2) for fluorescent silk production in silkworm transgenesis.

FIG. 3 provide white-light and fluorescent images of eCFP, eGFP, eYFP and mKate2 silk cocoons produced by silkworm transgenesis via the piggyBac transposase method.

FIGS. 4 and 5 are graphs of normalized absorption and normalized intensity vs. wavelength in nm, respectively, for eCFP, eGFP, eYFP and mKate2.

FIG. 6 is a schematic diagram of silk protein regeneration and edible PUF device fabrication using fluorescent silk (i.e. eCFP, eGFP, eYFP, and mKate2 silk) and natural white silk, according to the present disclosure.

FIG. 7 is bar graph of size distribution of the fluorescent silk microparticles used in edible PUFs, according to the present disclosure

FIG. 8 is a photograph of 30 different edible PUF devices used to characterize the overall PUF performance, according to the present disclosure.

FIG. 9 is a series of fluorescent intensity vs. time (sec) are shown depicting temporal photostability of eCFP, eGFP, eYFP, and mKate2 silk microparticles under excitation of 440 nm for eCFP, eGFP, and eYFP silk; 514 nm for mKate2 silk.

FIGS. 10a and 10b are two photographs depicting scalability (FIG. 10a) and flexibility (FIG. 10b) of the proposed edible PUFs for mass production and easy integration with medicines.

FIG. 11 is a schematic diagram of an exemplary customized imaging setup according to the present disclosure for obtaining fluorescent images of edible PUF devices.

FIG. 12 provides graphs of probability vs. normalized hamming distance (HD) to show intra-device (reproducibility) and inter-device (uniqueness) variabilities with a cut-off threshold of 0.1808.

FIG. 13 is a schematic showing feasibility of on-dose (or in-dose) authentication using edible PUFs.

FIG. 14 provides a series of photographs to depict the water solubility of edible PUFs composed of silk proteins and fluorescent proteins, according to the present disclosure.

FIG. 15 is a schematic of a concept of on-dose authentication, where each individual medicine in a solid oral dosage form (e.g. tablets and capsules) is integrated with an edible PUF device by the pharmaceutical manufacturer.

### DETAILED DESCRIPTION

The patent or application file contains at least one drawing executed in color. Copies of this patent or patent application publication with color drawing(s) will be provided by the Office upon request and payment of the necessary fee.

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

One excellent way for guaranteeing high security of on-dose authentication and protection against counterfeiting medicines is to utilize physically unclonable functions (PUFs). A PUF depends on the uniqueness of its physical microstructure that defines the PUF. This uniqueness depends on a myriad random physical factors introduced during manufacturing; and given that these factors are unpredictable and uncontrollable, duplication is substantially impossible. A PUF does not use a single encryption key that can possible be decoded and used without authorization. Instead, a PUF implements a challenge-response authentication to authenticate the associated microstructure. When a physical stimulus is applied to the structure, while it reacts in an unpredictable way, it reacts in a repeatable fashion. The applied stimulus is called a challenge, and the reaction of the PUF is called the associated response. Such a specific challenge and its corresponding response are held in a secure database, and thus authentication can be checked against such a database. The challenge-response and its communication with the secured database can be encrypted for added security.

Importantly, PUFs can be asymmetric such that it is easy to make a PUF, but is extremely challenging for counterfeiters to create a clone. Once an output response is read from the database, it cannot be re-used as well. The information on dose, frequency, and caution can be encoded for user adherence by labeling individual medicines.

For digital on-dose PUFs, the present disclosure presents silk proteins and fluorescent proteins as edible and digestible photonic biomaterials. From an edible perspective, important considerations include digestibility and nonallergenic properties. To this end, endogenous natural materials or biomaterials are chosen for on-dose applications. Importantly, silk proteins (i.e. fibroin) have excellent intrinsic functionality, biocompatibility, and low immunogenicity with minimal inflammatory and immune responses. Naturally-derived silk fibroin, without any external treatment, are dissolved in an aqueous solution. Silk proteins are also degradable and the degradation rate is controllable by using different silk regeneration and fabrication methods. More relevantly, silk proteins are edible and digestible. In addition, fluorescent proteins have been introduced into the food supply from genetically modified food. The potential toxicity and allergenicity are minimal with ingestion of green fluorescent protein. When compared with common food allergens, fluorescent proteins do not have common allergen epitopes and are degraded during gastric digestion. From an engineering perspective, processing of silk proteins is readily available for constructing structures and patterns from nanoscale to microscale. In particular, the polymeric nature from silk proteins can easily be fabricated into a variety of types of rigid or flexible structures with tunable mechanical and optical properties. Furthermore, silk proteins containing recombinant fluorescent proteins can be produced by transgenesis of genetically engineered domesticated silkworms. Transgenes of a variety of fluorescent proteins can be expressed by germline transformation using the gene splicing piggyBac method. The hybridization method can yield transformed silkworms with multiple successive generations and produce fluorescent silk in large amounts.

To this end, the present disclosure provides all protein-based PUFs that generate cryptographic keys with interactive multiple challenge-response pairs for on-dose authentication and anti-counterfeiting of medicines. The edible PUFs are made from silk (i.e. Bombyx mori) protein microparticles that are genetically fused with different fluorescent proteins, including enhanced cyan fluorescent protein (eCFP), enhanced green fluorescent protein (eGFP), enhanced yellow fluorescent protein (eYFP), and mKate2 (far-red) fluorescent protein. Particulate fluorescent silk is embedded in a thin film of natural white silk proteins, which can be directly attached onto the surface of a medicine in a solid oral dosage form. The source of entropy is a randomly scattered (or broadcast) fluorescent microparticle admixture of eCFP, eGFP, eYFP, and mKate2 silk, as the behavior of granular or particulate materials intrinsically has complex spatiotemporal fluctuations during the fabrication process. For the challenge-response requirement of PUFs, a unique set of excitation and emission bands of different fluorescent proteins serves as input challenges. The edible PUFs produce genuinely inherent output response images of spontaneous emission (i.e. fluorescence). The reported PUFs have strong randomness that guarantees unique and unpredictable cryptographic keys with a relatively large encoding capacity. We further characterize the fundamental PUF functions, including uniformity, uniqueness (for security), and reproducibility (for reliability), by calculating inter-device Hamming Distances (HDs), intra-device HDs, false positive rates, and false negative rates. The reported protein-based PUFs can provide an immediate solution with high security for on-dose authentication and anti-counterfeiting of medicines.

Fluorescent silk produced by the genetic fusion of fluorescent proteins (i.e. eCFP, eGFP, eYFP, and mKate2) via germline transformation (i.e. piggyBac transposon) and natural white silk were utilized. The following chemicals were used; dialysis tube (pore size 12,000 Da MWCO), lithium bromide (LiBr, ≥99%), miracloth (pore size 22-25 μm), sodium carbonate (Na₂CO₃,>99%), and Triton X100, purchased from Sigma-Aldrich Co. (Milwaukee, WI, USA). To select appropriate particle sizes and to broadcast fluorescent silk microparticles, we used two standard test sieves with opening sizes of 90 (No. 170) and 106 (No. 140) um, purchased from Cole-Parmer (Niles, IL, USA). All experiments were performed under the ambient conditions (22+2° C. and 40+10% relative humidity). It should be noted that any organic solvents and synthetic polymers were completely avoided for edibility and safe consumption.

A variety of image processing techniques can be used to extract information from the PUF and to securely check against the database for authentication. One such novel technique is described in the sister patent application filed on the same day as the instant patent application, entitled: IMAGE PROCESSING AND AUTHENTICATION OF UNCLONABLE FUNCTIONS.

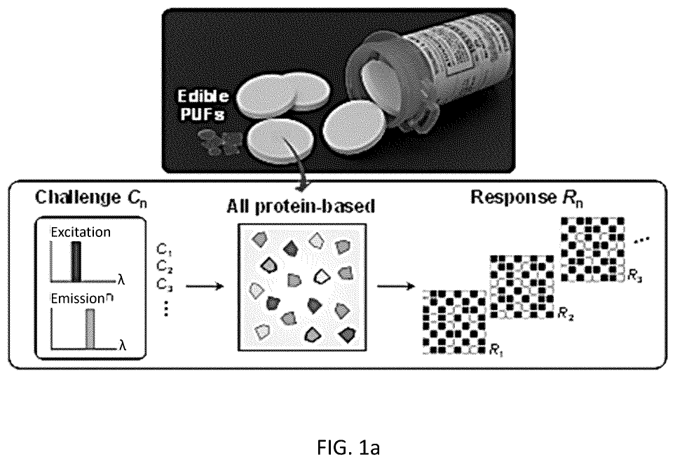

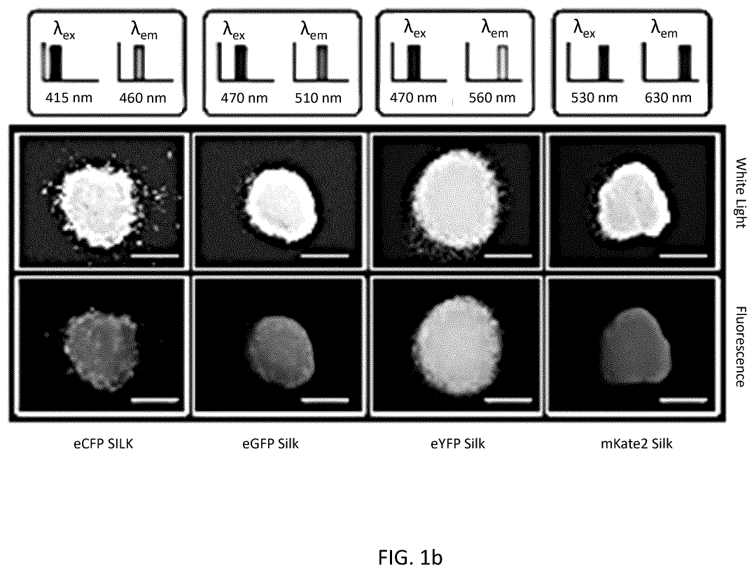

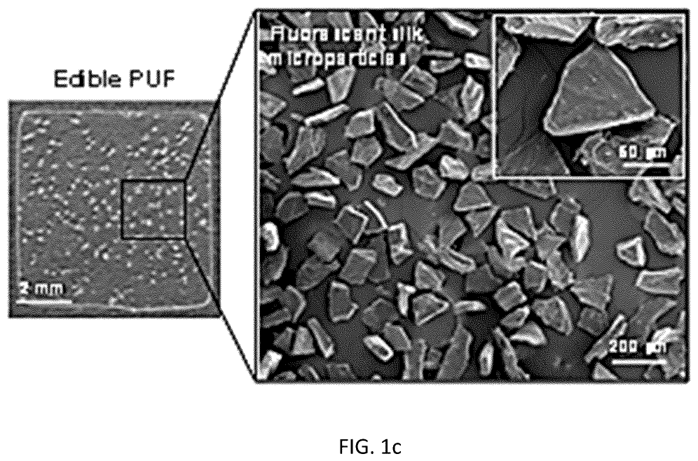

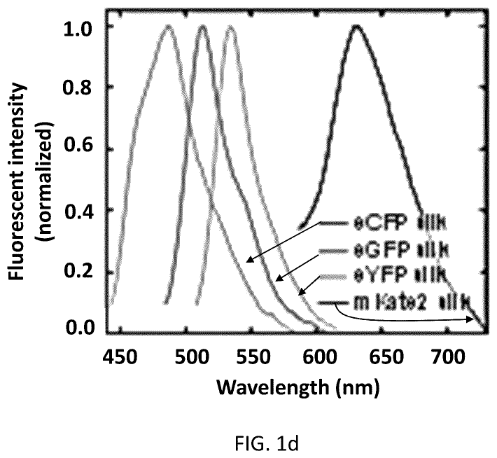

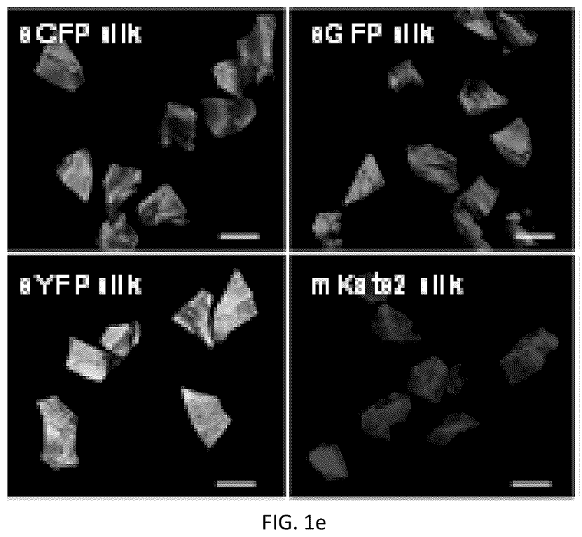

Referring to FIG. 1a, a schematic of edible PUFs for use with pharmaceuticals is shown. FIG. 1a shows a schematic illustration of an on-dose PUF with a photograph of covert and transparent PUFs attached on the surface of medicines. The PUF device is composed of proteins from fluorescent proteins and silk that are edible and digestible. The distinct photoluminescent properties of fluorescent proteins in silk provide the parametric support of unique challenge-response pairs. In reaction by an input challenge, the edible PUF generates its corresponding output response. FIG. 1b shows that the photoluminescent properties of fluorescent silk proteins are used to realize multiple challenge-response pairs in an edible PUF platform for heightened security. FIG. 1c is a photograph of an edible PUF device in which fluorescent silk microparticles are embedded in a thin silk film. SEM image of fluorescent silk microparticles with zeolite-like shapes is shown. FIG. 1d is an emission spectra of particulate eCFP, eGFP, eYFP and mKate2 silk which cover a relatively broad wavelength range in the visible light, while the emission peak positions are not overlapped among others. FIG. 1e are confocal fluorescence microscopy images of the corresponding fluorescent silk microparticles under excitation of 405, 458, 514, or 561 nm. The scale bar is 100 μm. The size of fluorescent silk microparticles is 99.3+7.9 μm (mean±standard deviation). Importantly, challenge-response pairs differentiate the protein-based PUFs of the present disclosure from other common unique objects and tags. In reaction to optical challenges, defined by a unique set of excitation and emission bands of different fluorescent proteins, the edible PUF made of silk protein (i.e. fibroin) and fluorescent proteins generates distinct output responses. The source of entropy is randomly distributed fluorescent silk microparticles seamlessly embedded in a covert thin transparent silk film. First, we take advantage of four different fluorescent proteins (i.e. eCFP, eGFP, e YFP, and mKate2) that have specific excitation and emission peaks in the visible wavelength range (provided in Table 1).

TABLE 1
Optical properties of fluorescent proteins
genetically hybridized with silk

| | Excitation | Emission | Extinction | |
|---|---|---|---|---|
| Fluorescent | maximum | maximum | coefficient | Quantum |
| protein | (nm) | (nm) | (M⁻¹ cm⁻¹) | yield (%) |
| eCFP | 434 | 477 | 32,500 | 40 |
| eGFP | 489 | 509 | 55,000 | 60 |
| eYFP | 514 | 527 | 84,000 | 61 |
| mKate2 | 588 | 633 | 62,500 | 40 |

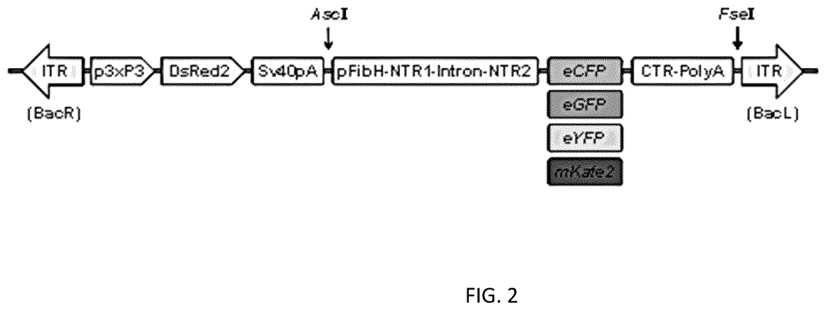

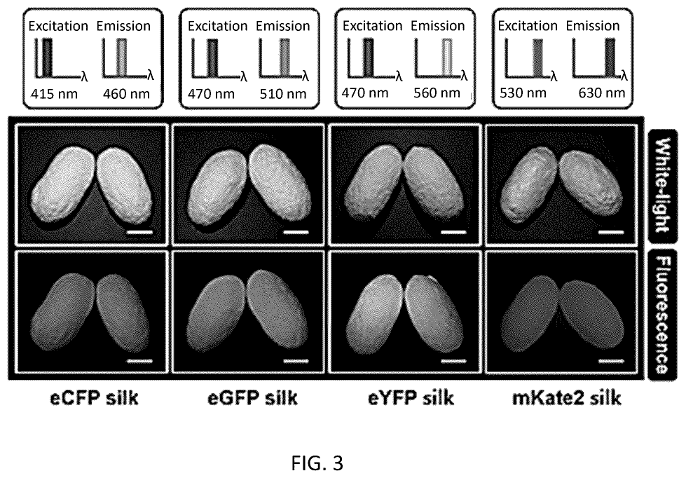

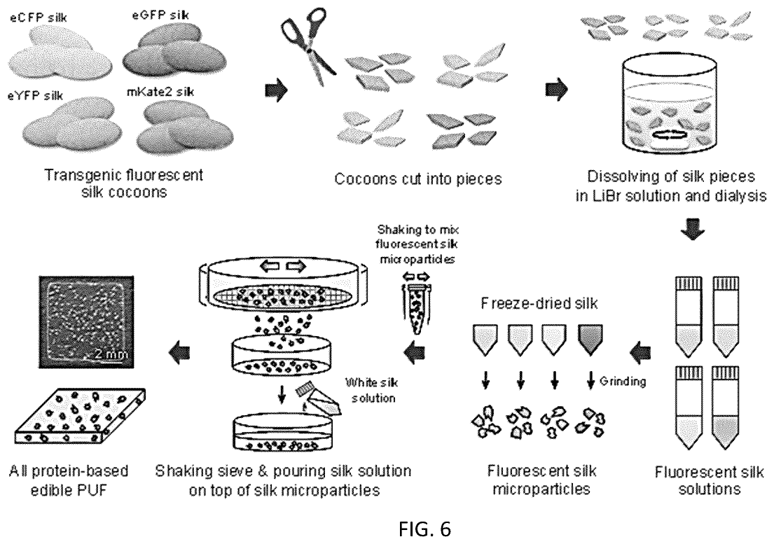

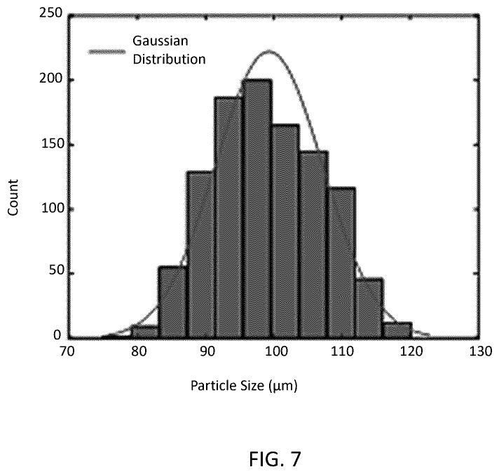

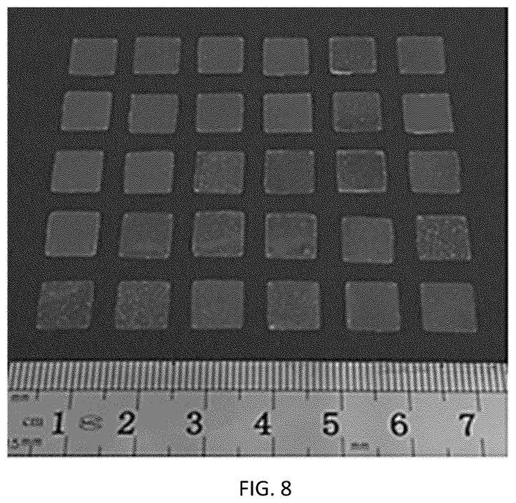

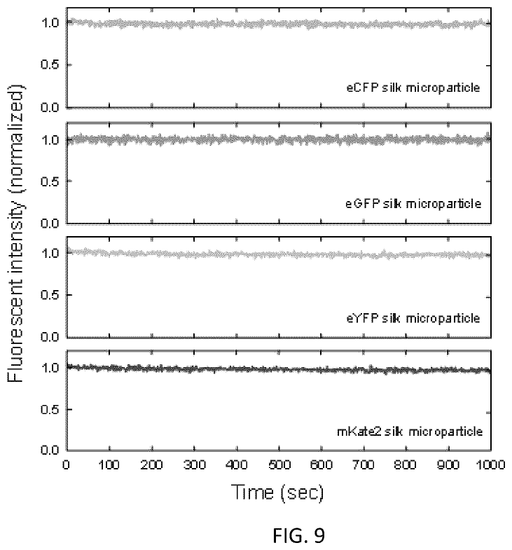

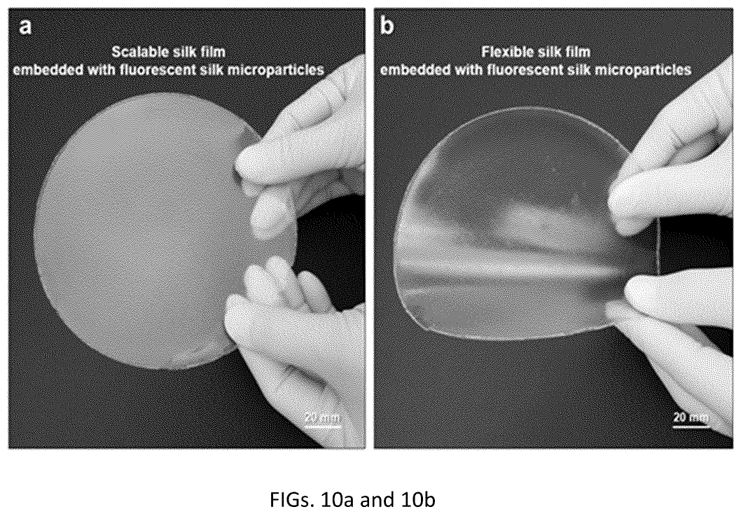

Specifically, we utilize fluorescent protein-expressed silk produced by transgenic silkworms as recombinant proteins via the piggyBac transposase method (see FIG. 2, discussed below). Silk proteins are an excellent biopolymer to be genetically hybridized with fluorescent protein genes. Second, to fabricate fluorescent silk microparticles (see FIG. 6, discussed below), fluorescent silk fibroin is regenerated into an aqueous solution with a low-temperature process, is freeze-dried, and is gently ground into zeolite-shaped microparticles with sizes of 99.3+7.9 μm (mean±standard deviation) (see FIGS. 1b and 1c and FIG. 7, discussed below). Third, an admixture of the fluorescent silk microparticles is broadcast on a large flat surface and a white silk fibroin solution is poured on top. After an ambient drying process in the dark, this thin transparent silk film with a thickness of 150 μm is punched into 7×7 mm² squares, resulting in all protein-based edible PUF devices (see FIGS. 6 and 8 discussed below). eCFP, eGFP, eYFP, and mKate2 silk cocoons possess bluish, greenish, yellowish, and reddish colors under white light illumination, respectively (see FIG. 3, discussed below). However, after the regeneration of the fluorescent silk, each type of fluorescent silk microparticles are not distinguishable with the naked eye, while maintaining their fluorescent properties (see FIGS. 1b-1e and FIG. 9, discussed below). This fabrication process is scalable for mass production without using any sophisticated equipment and is safe for oral consumption without any organic solvents or synthetic polymers (e.g. methanol, ethanol, isopropanol, or polyvinyl alcohol) (see FIG. 10, discussed below).

Referring to FIG. 1b, regenerated particulate eCFP, eGFP, e YFP and mKate2 silk produced by silkworm transgenesis via the piggyBac transposase method in which fluorescent proteins and silk (i.e. Bombyx mori) are genetically fused as recombinant proteins are shown. In FIG. 1b, the fluorescent images are acquired with the set of the excitation (λₑₓ) and emission (λₑₘ) wavelengths, as specified on top of each photograph. The scale bar is 5 mm. FIG. 1c provides photograph of an edible PUF device in which fluorescent silk microparticles are embedded in a thin silk film. SEM image of fluorescent silk microparticles with zeolite-like shapes. FIG. 1d provides the emission spectra of particulate eCFP, eGFP, eYFP and mKate2 silk which cover a relatively broad wavelength range in the visible light, while the emission peak positions are not overlapped among others. FIG. 1e is confocal fluorescence microscopy images of the corresponding fluorescent silk microparticles under excitation of 405, 458, 514, or 561 nm. The scale bar is 100 μm. The size of fluorescent silk microparticles is 99.3+7.9 μm (mean±standard deviation), as shown in FIG. 7, as discussed below.

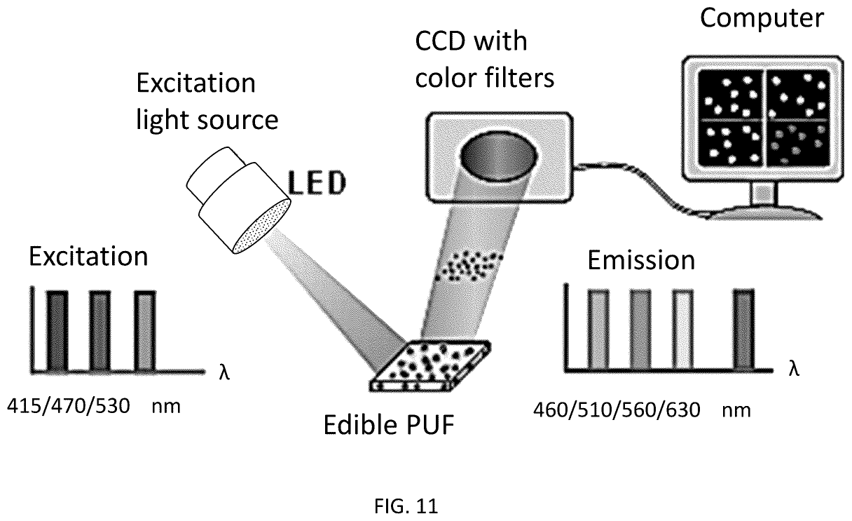

According to the present disclosure a cryptographic key is extracted from an output response when optically challenged, including the raw output measurement, the bitstream extraction, and the final digitized security key. According to one embodiment, four representative challenge-response pairs (n=4) are used based on the excitation and emission peak wavelengths of the individual fluorescent proteins in silk. An input challenge (Cn) is selected as a combination of the excitation and emission bands at specific wavelengths such as λₑₓ=415 nm and λₑₘ=460 nm; λₑₓ=470 and λₑₘ=510 nm; λₑₓ=470 and λₑₘ=560 nm; λₑₓ=530 and λₑₘ=630 nm, corresponding to eCFP, eGFP, e YFP, and mKate2 in silk, respectively. Upon optical excitation, a raw fluorescent image is recorded by a charge-coupled device (CCD) camera equipped with a conventional zoom lens via a tunable color filter that can be used for a down-stream optical recognition arrangement (see FIG. 11, discussed below).

Referring to FIG. 3, white-light and fluorescent images of eCFP, eGFP, eYFP and mKate2 silk cocoons produced by silkworm transgenesis via the piggyBac transposase method are shown. The fluorescent images are taken with the set of the excitation (λₑₓ) and emission (λₑₘ) wavelengths, as specified on each photograph. The scale bar is 10 mm.

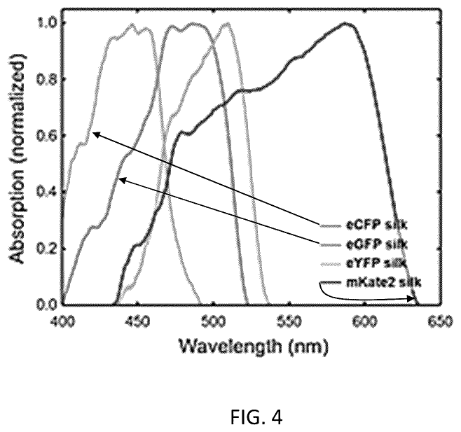

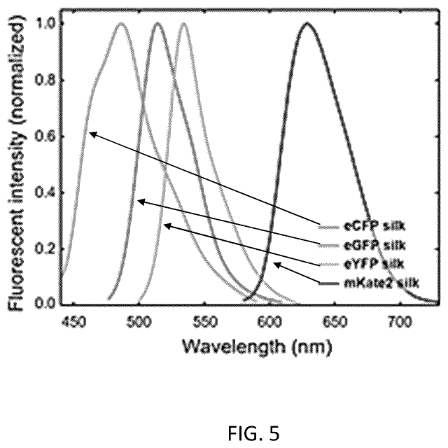

Referring to FIGS. 4 and 5, normalized absorption and fluorescence spectra of eCFP, eGFP, eYFP and mKate2 transgenic silk are shown. The excitation wavelengths are 415, 470, 470, and 530 nm for eCFP, eGFP, eYFP and mKate2 silk, respectively.

Referring to FIG. 6, a schematic diagram of silk protein regeneration and edible PUF device fabrication using fluorescent silk (i.e. eCFP, eGFP, eYFP, and mKate2 silk) and natural white silk is shown, according to the present disclosure. Construction of plasmid vector DNA for silkworm transgenesis is first discussed. Using germline transformation (i.e. piggyBac transposon), the transition vectors pBac-3xP3-DsRed2-pFibH-(eCFP, eGFP, or eYFP) and pBac-3xP3-eGFP-pFibH-mKate2 were constructed as the piggyBac-derived vector and the injected the vector DNA with a helper vector into pre-blastoderm embryos. Referring to FIG. 2, a transformation vector of p3xP3-DsRed2-FibH-(eCFP, eGFP, eYFP, or mKate2) for fluorescent silk production in silkworm transgenesis is shown. In the case of mKate2, eGFP is used instead of DsRed2. The nucleotide sequences of pFibH-NTR and CTR are derived from Genebank Accession No. AF226688. pFibH: fibroin heavy chain promoter domain (1124 bp), NTR1: N-terminal region 1 (142 bp), intron: first intron (871 bp), NTR2: N-terminal region 2 (417 bp), CTR: C-terminal region (179 bp), PolyA: poly(A) signal region (301 bp), eCFP: enhanced cyan fluorescent protein, eGFP: enhanced green fluorescent protein, eYFP: enhanced yellow fluorescent protein, mKate2: monomeric far-red fluorescent protein, DsRed2: red fluorescent protein (a mutant form of DsRed from Discosoma sp.), ITR: inverted repeat sequences of piggyBac arms, 3xP3: 3xP3 promoter, and SV40: SV40 polyadenylation signal sequence. The restriction enzyme sites for the construction of recombinant vectors are indicated with the arrows For construction of plasmids, DsRed2 (eGFP for mKate2) cDNA as a marker was amplified by polymerase chain reaction (PCR) using specific primers with NheI/AflII sites from pDsRed2-C1 (NheI-DsRed2-F: 5′-GCTAGCATGGCCTCCTCCGAGAAC-3′ and DsRed2-AfIII-R: 5′-CTTAAGCTACAGGAACAGGTGGTGGCG-3′; Clontech, Mountain View, CA, USA) and then cloned into the pGEM-T Easy Vector System (Promega, Co.), designated as pGEMT-DsRed2. DsRed2 gene was excised from pGEM-DsRed2 digested with restriction enzymes of NheI/AflII and replaced with eGFP gene from pBac-3xP3-eGFP to form pBac-3xP3-DsRed2. To obtain fibroin promoter, a DNA fragment containing the promoter domain (1,124 bp) and N-terminal region (1,430 bp) with the intron (972 bp) of the fibroin H gene (GenBank Accession No. AF226688, nt. 61,312-63,870) was amplified by PCR using the genomic DNA from Bombyx mori and primers (pFibHN-F: 5′-GGCGCGCCGTGCGTGATCAGGAAAAAT-3′ and pFibHN-R: 5′-TGCACCGACTGCAGCACTA GTGCTGAA-3′), subsequently cloned into pGEM-T Easy Vector System. The resultant DNA fragment was named as pGEMT-pFibH-NTR. The DNA fragment containing 180 bp of the 3′ terminal sequence of the H-chain gen open reading frame (ORF) and 300 bp of the 3′ region of the fibroin H gen (GenBank Accession No. AF226688, nt. 79,021-80,009) was amplified by PCR using genomic DNA from Bombyx mori and primers (pFibHC-F: 5′-AGCGTCAGTTACG GAGCTGGCAGGGGA-3′ and pFibHC-R: 5′-TATAGTATTCTTAGTTGAGAAGGCATA-3′) and then was cloned into pGEM-T Easy Vector System. This fragment is designated as pGEMT-CTR. The fragments were prepared by digesting pGEMT-pFibH-NTR with AscI/SalI and pGEMT-CTR with SalI/SacI, respectively. These two fragments were cloned with pBluescriptII SK (-) vector (Stratagene) digested with Apal/SalI, resulting in pFibHNC-null. Fluorescent genes (eCFP, eGFP, eYFP, and mKate2) were synthesized and purchased from the BIONEER corporation (South Korea). The N- and C-terminals had the NotI and Sbfl restriction sites, respectively. The fluorescent genes were digested with NotI/Sbfl and subcloned into a pFibHNC-null, resuling in pFibHNC-eCFP, pFibHNC-eGFP, pFibHNC-eYFP, and pFibHNC-mKate2, respectively. The resultant vector was named as pBac-3xP3-eCFP-FibH, pBac-3xP3-DsRed2-FibH-eGFP, pBac-3xP3-DsRed2-pFibH-eYFP, and pBac-3xP3-eGFP-FibH-mKate2, respectively.

To avoid heat-induced denaturation of fluorescent proteins in silk, we performed a dissolution process of fluorescent silk under a low temperature. Sericin of transgenic silk cocoons was removed with minimizing heat-induced denaturation of fluorescent proteins (i.e. eCFP, eGFP, eYFP, and mKate2). The silk cocoons were treated several times with a mixture solution of Na₂CO₃ (0.2%) at temperatures lower than 50° C., subsequently rinsed with warm deionized water (˜ 35° C.) several times. During a degumming process, the low pressure (620 mmHg) was additionally treated to completely remove the sericin. After degumming, the sericin-removed cocoons were dried under dark ambient conditions. For white silk, we used a conventional degumming method under a boiling process. It should be noted that Na₂CO₃ (also known as soda crystals) is an inactive ingredient for drug products approved by FDA. In addition, it is well known that natural silk also contains the elements of carbon (C), oxygen (O), and sodium (Na).

After sericin was removed, fluorescent silk cocoons were cut into small pieces with sizes less than 2-5 mm and then were dissolved in an aqueous solution of LiBr (9.5 M) at 45° C. for four hours with stirring of 400 rpm. The dissolved solution was filtered through a miracloth and was dialyzed in deionized water at room temperature for about two days with a cellulose semipermeable tube to remove the salt (i.e. LiBr). For eCFP, eGFP, eYFP, and mKate2 fluorescent silk, each regenerated silk fibroin solution with a concentration of about 5% (w v-1) was obtained after centrifuging with a speed of 9000 rpm at about 4° C. for about 20 minutes. The regenerated solutions were freeze-dried at about −18° C. for seven days. The freeze-dried fluorescent silk was mechanically ground into granular microparticles with a mortar and pestle. Fluorescent silk microparticles with a size range of 90-106 μm were selected by shaking them through a stack of two standard test sieves with opening sizes of 90 and 106 μm. Similarly, a white silk fibroin solution was prepared in a dissolving solution of LiBr (9.5 M) at 60° C. for four hours with stirring of 400 rpm, resulting in a concentration of 4% (w v-1). It was filtered through a miracloth and was dialyzed in deionized water at room temperature for about two days with a cellulose semipermeable tube, followed by centrifugation with a speed of 9000 rpm at about 4° C. for about 20 minutes.

To fabricate edible PUF devices, fluorescent silk microparticles were mixed at a ratio of 1:1:1:1 (eCFP, eGFP, eYFP, and mKate2 silk) in a microcentrifuge tube and then was shaken by hand. The admixture of the microparticles was broadcast to the surface of a polystyrene Petri dish (diameter 35 mm) through a sieve with an opening size of 106 μm by mechanical shaking. Subsequently, the natural white silk fibroin solution of 4 mL was poured on the plastic petri dish and was cast at ambient conditions (25+2° C. and 40-50% relative humidity) in the dark for three days, resulting in a silk film with a thickness of about 150 μm, including four different fluorescent silk microparticles. Edible PUFs were prepared by punching the silk films with a square area of 7×7 mm² (see FIG. 8, discussed below).

As easily accessible common light sources for optical excitation, we used ultraviolet, blue, and green light-emitting diodes (LEDs) with emission wavelengths of 415 nm (FWHM=14 nm), 470 nm (FWHM=25 nm), and 530 nm (FWHM=33 nm) purchased from Thorlabs Inc. (Newton, NJ, USA). Bandpass filters of 410, 470, and 530 nm (FB410-10, FB470-10, and FB530-10; Thorlabs Inc.) were placed between the light source and the PUF device. The optical power was kept at 1, 3, and 10 mW mm-2 for 415-nm, 470-nm, and 530-nm LEDs at the surface of PUFs, respectively. To image the PUFs, we used a charge-coupled device (CCD) camera (Princeton Instruments PIXIS 1024B) with a conventional zoom lens (MVL7000, Navitar, Rochester, NY, USA) via a liquid crystal tunable filter with a FWHM of 7 nm (VariSpec VIS-07-20; PerkinElmer, Inc., Waltham, MA, USA). As a result, the following set of excitation and emission bands was selected such that λₑₓ=415 nm and λₑₘ=460 nm; λₑₓ=470 and λₑₘ=510 nm; λₑₓ=470 and λₑₘ=560 nm; λₑₓ=530 and λₑₘ=630 nm, optimized for eCFP, eGFP, eYFP, and mKate silk, respectively.

According to one embodiment of the present disclosure, during the regeneration process for the fabrication of colored silk particles, edible fluorescent color dyes (Table 2) can be mixed with a dissolved silk protein solution or an edible polymer solution. Specifically, edible fluorescent color dyes can be used as a color source for the realization of colored silk particles in addition to transgenic fluorescent silk. The color dyes can be easily dissolved in water and integrated with silk proteins or polymers during the regeneration process.

Referring to FIG. 7, a bar graph of size distribution of the fluorescent silk microparticles used in edible PUFs is shown, according to the present disclosure. The particle sizes are characterized from SEM images. A Gaussian fit returns a mean of 99.3 μm and a standard deviation (SD) of 7.9 μm. Referring to FIG. 8, a photograph of 30 different edible PUF devices used to characterize the overall PUF performance is provided, according to the present disclosure. Each size is 7×7 mm². Referring to FIG. 9, a series of fluorescent intensity vs. time (sec) are shown depicting temporal photostability of eCFP, eGFP, eYFP, and mKate2 silk microparticles under excitation of 440 nm for eCFP, eGFP, and eYFP silk; 514 nm for mKate2 silk. The emission intensity is measured through a 458-nm lowpass filter for eCFP, eGFP, and eYFP silk; a 550-nm lowpass filter for mKate2 silk over 1000 seconds. Referring to FIGS. 10a and 10b, two photographs are shown depicting scalability (FIG. 10a) and flexibility (FIG. 10b) of the proposed edible PUFs for mass production and easy integration with medicines.

Referring to FIG. 11, a schematic diagram of an exemplary customized imaging setup according to the present disclosure is provided for obtaining fluorescent images of edible PUF devices. As easily accessible common light sources for optical excitation, ultraviolet, blue, and green light-emitting diodes (LEDs) at central wavelengths of 415, 470, and 530 nm are used. Fluorescent images are acquired through a liquid crystal tunable filter with emission wavelengths of 460, 510, 560, and 630 nm with acquisition times of 60, 5, 40, and 30 sec, respectively. Different combination sets of the excitation and emission wavelengths serve as challenges to realize the parametric support of challenge-response pairs in edible PUFs. Given some high-end smartphones already have multiple light sources (e.g. flashlight LED), we envision that multiple different colored LEDs can easily be embedded into a smartphone as manufacturers realize a variety of direct applications, including the proposed on-dose authentication.

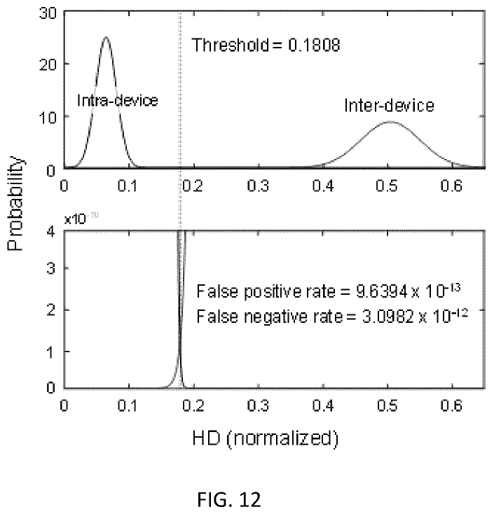

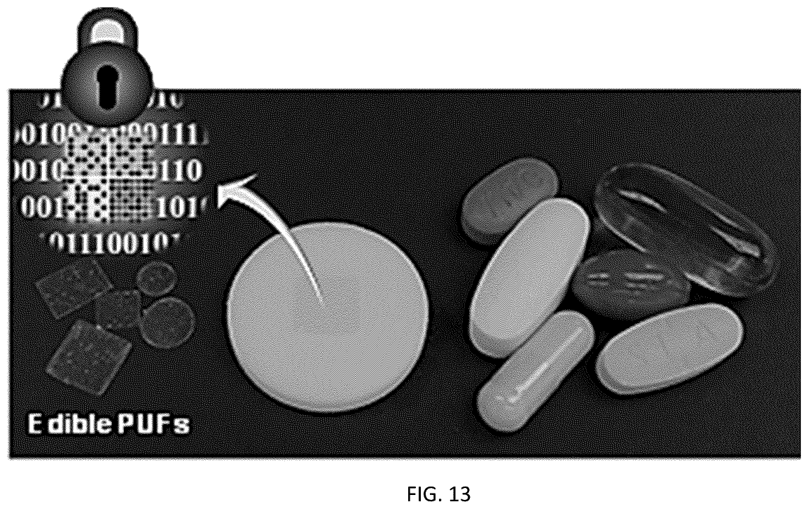

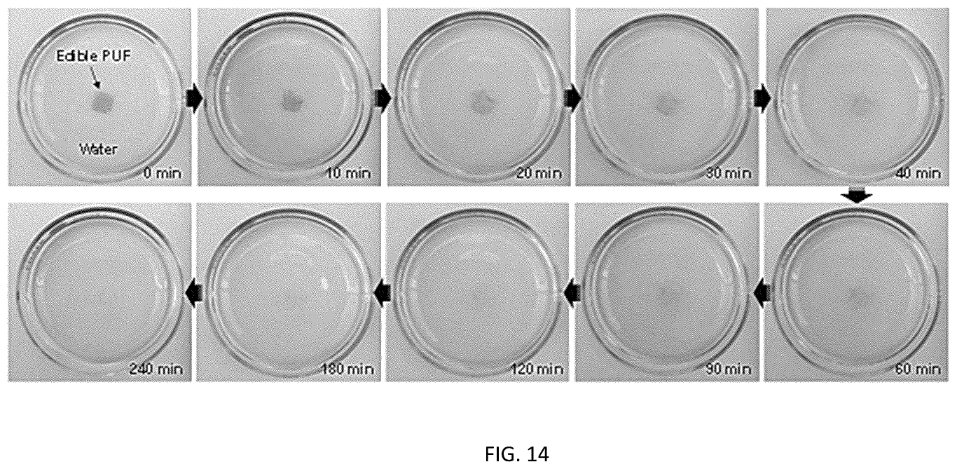

Referring to FIG. 12, graphs of probability vs. normalized hamming distance (HD) are provided to show intra-device (reproducibility) and inter-device (uniqueness) variabilities with a cut-off threshold of 0.1808. The resulting false positive and false negative rates are 9.6394x10-13 and 3.0982×10-12, respectively. Referring to FIG. 13, a complex photograph of pharmaceuticals is shown to depict feasibility of on-dose (or in-dose) authentication using edible PUFs, according to the present disclosure. Referring to FIG. 14, a series of photographs is shown to depict the water solubility of edible PUFs composed of silk proteins and fluorescent proteins, according to the present disclosure. The natural silk fibroin film embedded with eCFP, eGFP, eYFP, and mKate2 silk microparticles (loaded with methylene blue for easy visualization purpose) in deionized water is completely dissolved in 240 minutes at room temperature.

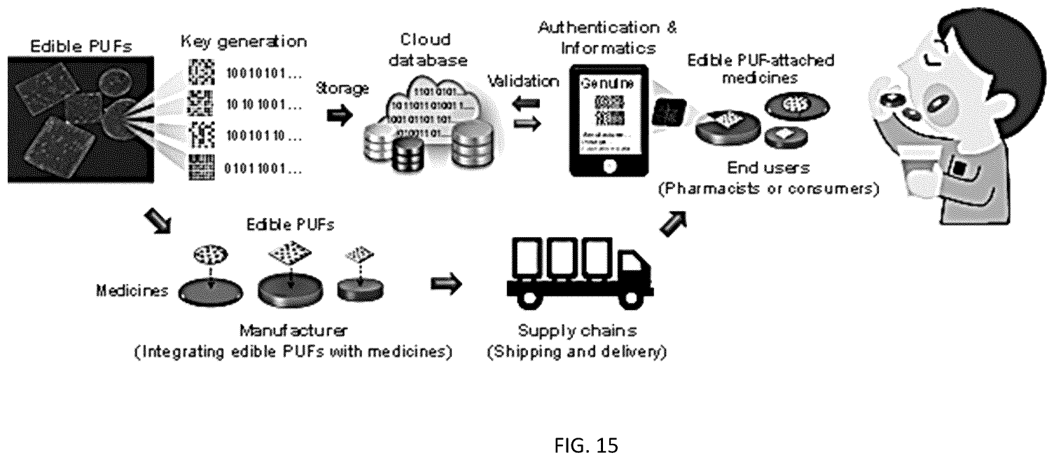

One prominent application of the reported edible PUFs is on-dose authentication to prevent patients from taking counterfeit pharmaceutical products. Referring to FIG. 15, an exemplary schematic of the process flow representing such authentication is shown. The edible PUF, which is flexible (see FIG. 10b), can be attached to the surface of medicines in a solid oral dosage form including pills, tablets, and capsules. Each medicine possesses unique challenge-response pairs and the end user can verify genuine or fake using a smartphone camera or a customized reader and accessing the registered digital keys in a database (e.g. cloud) where each validation is guided with a trusted authority against the digital identity. Indeed, the edible PUF has a self-vanishing feature. Silk proteins (i.e. fibroin) are easily be dissolved in an aqueous solution without any special treatments, owing to the disintegration property and proteolytic activity (i.e. enzymatic degradation). When the reported edible PUF is loaded with a blue dye (i.e. methylene blue) for easy visual detection purpose, it is completely dissolved in deionized water after 240 minutes (see FIG. 14), also supporting the use for oral consumption. In other words, the end user (i.e. patient) can take the medicine without removing the PUF from the surface.

It should be appreciated that in addition to the material discussed herein, other materials including edible polymers, edible proteins, and edible dyes can be used for the PUF. For example, one or more of the following edible polymers can be used in the PUF of the present disclosure: the edible polymers are selected from the group consisting of Starch, cellulose derivatives, chitosan, pectin, alginates, gums, carrageenans, and combinations thereof. In addition, one or more of the following edible polymers can be used in the PUF of the present disclosure: gelatin, collagen, albumin, milk protein, and combinations thereof. In addition, one or more of the following edible polymers can be used in the PUF of the present disclosure: zein, soy, wheat gluten, lectins, and combinations thereof. In addition, one or more of the following edible polymers can be used in the PUF of the present disclosure: Fatty acids, triglycerides, phospholipids, and combinations thereof. In addition, one or more of the following edible fluorescent proteins can be used in the PUF of the present disclosure: red fluorescent protein (DsRed), orange fluorescent protein (mKO), and a combination thereof. In addition, one or more of the following edible fluorescent dyes can be used in the PUF of the present disclosure: Brilliant Blue FCF, Indigotine, Fast Green FCF, Erythrosine, Allura Red AC, Tartrazine, Sunset Yellow FCF, and combinations thereof, as provided in Table 2, below.

TABLE 2
Food dye colorings approved by the U.S. Food and Drug Administration.
FD&C stands for laws passed by the U.S. Congress in 1938,
called the Federal Food, Drug, and Cosmetic Act.

| | | | Excitation/ | |
|---|---|---|---|---|
| | | | Emission | |
| FD&C | | | wavelengths | |
| Designation | Name | Color | [1] | Molecular Formula |
| Blue No. 1 | Brilliant | Blue | 580-600 nm/ | C₃₇H₃₄N₂Na₂O₉S₃ |
| | Blue FCF | | 650-700 nm | |
| Blue No. 2 | Indigotine | Indigo | — | C₁₆H₈N₂Na₂O₈S₂ |
| Green No. 3 | Fast Green FCF | Turquoise | 580-600 nm/ | C₃₇H₃₄N₂Na₂O₁₀S₃ |
| | | | 650-700 nm | |
| Red No. 3 | Erythrosine | Pink | — | C₂₀H₆I₄Na₂O₅ |
| Red No. 40 | Allura Red AC | Red | 450-540 nm/ | C₁₈H₁₄N₂Na₂O₈S₂ |
| | | | 540-610 nm | |
| Yellow No. 5 | Tartrazine | Yellow | 450-540 nm/ | C₁₆H₉N₄Na₃O₉S₂ |
| | | | 540-610 nm | |
| Yellow No. 6 | Sunset | Orange | 450-540 nm/ | C₁₆H₁₀N₂Na₂O₇S₂ |
| | Yellow FCF | | 540-610 nm | |

It should be appreciated that according to the present disclosure, particles are applied either i) to a substrate or ii) to a pharmaceutical directly. The substrate can be made of an edible silk or an edible polymer, according to the present disclosure. The particles can be one or more of edible silk (e.g., edible fluorescent silk), edible dyes (e.g., edible fluorescent dyes), edible polymers, according to the present disclosure. These particles can be cast into a random pattern onto either the substrate, or the pharmaceutical directly. Alternatively, these particles can be sprayed onto either the substrate, or the pharmaceutical directly in order to generate a random pattern. Once the particles are applied, an image can be obtained from these particles and a cryptographic key generated representing an original authentication pattern. This cryptographic key is then stored in a secured database awaiting authentication by an end user. The image can be obtained at the source of the pharmaceutical by a single image capture device representing an X-Y cryptographic key (i.e., a two-dimensional image) or by more than one image capture device (e.g., stereo-photography) representing an X-Y-Z cryptographic key (i.e., a three-dimensional image for a pharmaceutical with curvatures in the Z-direction).

According to another embodiment, the anti-counterfeit measures can directly be incorporated using spray deposition as an additive manufacturing process, resulting in security key-embedded pharmaceutical products. To support the unclonability (i.e. asymmetricity), it is critical to include a source of entropy (physical disorder) in a scalable manner. In other words, a spray system serves as a scalable entropy source for randomness. Such spray deposition of silk microparticles have unpredictable variations due to the stochastic nature of atomization as well as the impingement dynamics. Even with the same spraying process, it is virtually impossible to duplicate or clone the previously generated patterns. Thus, this manufacturing approach makes the PUF printing more cost-effective and scalable.

Towards this end, solid silk microparticles are deposited with a low density on the surface of medicines in a scalable manner with a commercially available cold spray system. In cold spraying, solid microparticles (powders) are accelerated in a supersonic gas jet. During impact with the substrate, microparticles undergo plastic deformation and adhere to the surface. Silk microparticles are fed from the divergent section of the nozzle and are accelerated by a driving gas to the target surface. In doing so, the microparticles are impregnated onto the surface of the pharmaceutical and thus fixed in place. Once fixed, these microparticles can be challenged by light to generate a response in the form of a cryptographic key that can be recorded in a remote database for later confirmation. This confirmation is based on application of light at different wavelengths at later time, which will generate a cryptographic pattern that can be checked against the cryptographic key.

Those having ordinary skill in the art will recognize that numerous modifications can be made to the specific implementations described above. The implementations should not be limited to the particular limitations described. Other implementations may be possible.

1. A method of fabricating and applying a physically unclonable function to a pharmaceutical, comprising:
constructing plasmid vector DNA for silkworm transgenesis to thereby generate one or more fluorescent silk proteins;
removing sericin from the one or more fluorescent silk proteins;
cutting the sericin-removed one or more fluorescent silk proteins into a plurality of pieces;
dissolving the cut pieces with a solution;
filtering the dissolved solution;
dialyzing the filtered dissolved solution;
centrifuging the dialyzed solution to thereby provide a regenerated silk fibroin solution;
freeze-drying the regenerated silk fibroin solution;
mechanically grinding the freeze-dried silk fibroin into granular silk microparticles;
sieving the mechanically ground silk fibroin to generate fluorescent silk microparticles;
generating an admixture of the fluorescent silk microparticles;
broadcasting the admixture of the fluorescent silk microparticles to the surface of a polystyrene Petri dish thereby generating a silk film that are adapted to be attached to a pharmaceutical,
wherein the silk film includes random distribution of the one or more fluorescent silk proteins.

2. The method of claim 1, wherein the one or more fluorescent silk proteins include eCFP, eGFP, eYFP, and mKate2.

3. The method of claim 2, the cleaning of the one or more fluorescent silk proteins is in a mixture solution of Na₂CO₃ (0.2%) at temperatures lower than 50° C.

4. The method of claim 3, wherein the cut sericin-removed one or more fluorescent silk proteins range from about 2 mm to about 5 mm.

5. The method of claim 4, wherein the cut pieces are dissolved in an aqueous solution of LiBr (9.5 M) at 45° C. for four hours with stirring of 400 rpm.

6. The method of claim 5, wherein the dissolved solution was filtered through a miracloth.

7. The method of claim 6, wherein the filtered solution was dialyzed in deionized water at room temperature for about two days with a cellulose semipermeable tube to remove the LiBr.

8. The method of claim 7, wherein the centrifuging of the dialyzed solution is with a speed of 9000 rpm at about 4° C. for about 20 minutes to thereby form the regenerated silk fibroin solution.

9. The method of claim 8, wherein the regenerated silk fibroin solutions were freeze-dried at about −18° C. for seven days.
